(19) United States
(12) Patent Application Publication
(10) Pub. No.: US 2009/0258343 A1
(43) Pub. Date: Oct. 15, 2009

(54) Semi-quantitative immunochromatographic device and method for the determination of HIV/AIDS immune-status via measurement of soluble CD40 Ligand/CD154, A CD4+ T cell equivalent and the simultaneous detection of HIV infection via HIV antibody detection

(76) Inventor: Paul C. Reiter, East Hampstead, NH (US)

Correspondence Address: BODNER & O'ROURKE, LLP, 425 BROADHOLLOW ROAD, SUITE 108, MELVILLE, NY, 11747, US

(21) Appl. No.: 12/454,951

(22) Filed: May 27, 2009

Related U.S. Application Data
(63) Continuation-in-part of application No. 12/288,180, filed on Oct. 17, 2008.
(60) Provisional application No. 60/981,110, filed on Oct. 19, 2007.

(51) Int. Cl. C12Q 1/70; C12M 1/00

(52) U.S. Cl. 435/5; 435/287.2

(57) ABSTRACT

A semi-quantitative, immunochromatographic dual test device for the simultaneous detection of HIV/AIDS immune status CD4+ T cell equivalents, such as soluble CD40 ligand/CD 154, and the detection of an HIV antibody, includes one or more support materials capable of providing lateral flow. The one or more support materials include at least one sample receiving area for receiving a biological sample containing a first target analyte, the first target analyte being a CD4+ T cell equivalent, such as soluble CD40 ligand/CD 154, and a second target analyte, the second target analyte being an HIV antibody. A second area, situated on the one or more support materials, has a movably contained detector ligand and or detector antigens, wherein the detector ligand and or detector antigens is capable of forming a mobile complex with the soluble CD40 ligand/CD 154 and or HIV antibodies, and at least a first capture area having a predetermined amount of a first immobile capture reagent, the first immobile capture reagent capable of specifically binding to the mobile complex formed by the soluble CD40 ligand/CD 154 protein and the detector ligand and providing a visible signal. The one or more support materials further have situated thereon at least a second capture area having a predetermined amount of a second immobile capture reagent that is capable of specifically binding to HIV antibodies present in the biological sample and providing a visible signal.

### CROSS-REFERENCE TO RELATED APPLICATIONS

This application is a continuation-in-part of U.S. application Ser. No. 12/288,180, filed on Oct. 17, 2008, and entitled “Semi-Quantitative Immunochromato graphic Device and Method for the Determination of HIV/AIDS Immune-Status Via Measurement of Soluble CD40 Ligand/CD 154, A CD4+ T Cell Equivalent”, the disclosure of which is incorporated herein by reference and on which priority is hereby claimed, which prior application is based on U.S. Provisional Application Ser. No. 60/981,110, filed on Oct. 19, 2007, and entitled, “Semi-Quantitative Immunochromatographic Device and Method For The Determination of HIV/AIDS Immune-Status Via Measurement of Soluble CD40 Ligand/CD 154, a CD4+ T Cell Equivalent”, the disclosure of which is also incorporated herein by reference and on which priority is also hereby claimed.

### BACKGROUND OF THE INVENTION

1. Field of the Invention

The present invention relates to a method and apparatus for monitoring immune system status and function, in HIV/AIDS patients, and for detecting HIV infection. More particularly, it relates to a new and improved device and method for the semi-quantitative detection, analysis and measurement of CD4+ T cells, through their surrogate marker, i.e., soluble CD40 ligand (CD 154); a protein expressed on the surfaces of CD4+ T cells following activation by HIV infection. Such semi-quantitative analysis and measurement in turn can be used to; stage HIV/AIDS patients to determine their immune status and whether anti-retroviral treatment should be initiated, or changed immediately. Furthermore, the device and method detects HIV infection via antibody detection.

2. Description of the Prior Art

There is a real need for new and better methods and apparatus for assaying CD4+ T cell levels in patients suffering from the Human Immunodeficiency Virus, irrespective of age, and irrespective of resources, particularly in third world countries where resources are scarce and men, women and children are dying in staggering numbers. Human Immunodeficiency Virus (HIV) is a retrovirus, i.e., a virus that contains two single-strand linear ribonucleic acid (RNA) molecules per virion and reverse transcriptase, which together allow the virus to transcribe RNA into DNA, instead of DNA to RNA. The virus-produced DNA in turn is incorporated into the host cell's DNA strand, which then produces new RNA retroviruses. In other words, the retrovirus restructures the host cell DNA to produce more retroviruses and thereafter kills the host cells. By killing the host cells, particular the host immunity producing cells, the retrovirus renders the host extremely vulnerable and leads to ACQUIRED IMMUNODEFICIENCY SYNDROME (AIDS), a condition in humans in which the immune system begins to fail, leading to life-threatening opportunistic infections.

Infection with HIV occurs by the transfer of blood, semen, vaginal fluid, pre-ejaculate, or breast milk. Within these bodily fluids, HIV is present as both free virus particles and virus infected immune cells. The four major routes of transmission are unprotected sexual intercourse, contaminated needles, and transmission from an infected mother to her baby at birth or through breast milk.

HIV primarily infects vital cells in the human immune system such as helper T cells and more specifically CD4+ T cells, macrophages and dendritic cells. HIV infection leads to low levels of CD4+ T cells through three main mechanisms: (i) via direct viral killing of infected cells; (ii) via increased rates of apoptosis in infected cells; and (iii) via the killing of infected CD4+ T cells by CD8 cytotoxic lymphocytes that recognize infected cells. When CD4 + T cells decline below a critical level, cell-mediated immunity declines and the body becomes progressively more susceptible to opportunistic infections. If untreated, eventually most HIV-infected individuals develop AIDS and die.

HIV infection in humans is now pandemic. As of January 2006, the Joint United Nations Programme on HIV/AIDS and the World Health Organization estimated that AIDS had killed more than 25 million people since it was first recognized on Dec. 1, 1981, making it one of the most destructive pandemics in recorded history. In 2005 alone, AIDS claimed an estimated 2.4-3.3 million lives, of which more than 570,000 were children. It is estimated that about 0.6% of the world's living population is infected with HIV.

A third of these deaths are occurring in sub-Saharan Africa, retarding economic growth and increasing poverty. According to the current estimates, HIV is set to infect 90 million people in Africa alone, resulting in a minimum estimate of 18 million orphans.

There is no cure for AIDS. On the other hand, there is anti-retroviral treatment (ART), which reduces both the mortality and the morbidity of HIV infection. The treatment consists of drugs that have to be taken every day for the rest of the patient's life, when the time is right. They work against the HIV infection itself by slowing down the replication of the HIV in the body.

It has been found that for antiretroviral treatment (ART) to be effective for a long time, it has been found that patients need to take more than one anti-retroviral drug at a time. When HIV replicates, i.e., makes new copies of itself, it often makes mistakes. This means that within any infected person there are many different strains of the HIV. Occasionally, a new strain is produced that happens to be resistant to the effect of one type of anti-retroviral drug. If the patient is not taking any other type of drug, then the resistant strain is able to replicate quickly and the benefits of the treatment are lost. Taking two or more anti-retrovirals at the same time vastly reduces the rate at which resistance develops.

However, there is a problem. The tools for the truly routine, truly inexpensive, speedy, yet effective determination of the need for and implementation of anti-retroviral treatment (ART) is just not available in any country.

It is well known that the progression of HIV/AIDS in HIV infected patients is monitored through CD4+ T cells. Specifically, the CD4+ T cell count in peripheral blood is used for: (i) assessing the degree of immune deterioration and speed of progression towards AIDS; (ii) defining, together with clinical information, decision points to initiate anti-retroviral treatment (hereinafter “ART”); (iii) deciding the time for prophylaxis of opportunistic infections; and (iv) monitoring the efficacy of treatment. Ideally, as soon as CD4 + T cells decline below internationally recognized critical levels, anti-retroviral treatment must be started immediately.

In other words, the CD4+ T cell count is a critical parameter in monitoring HIV disease. Specifically, measurements of CD4+ T cells are essential for staging HIV-infected patients, i.e. establishing the stage or level of the infection in HIV-positive patients. Once the stage or level of infection is established, the patients need for anti-retroviral medications can be ascertained, and the decision of whether anti-retroviral therapy should be implemented can be made with certainty. Once the anti-retroviral therapy is implemented, it can be monitored through the continued measurement of CD4+ T cells. Lower numbers of circulating CD4+ T cells imply a more advanced stage of HIV disease and less competent defense mechanisms.

In developed countries, CD4+ T cell counts are typically performed every three to six months for each patient using the method of flow cytometry. In fact, flow cytometry is considered the gold standard for the determination of CD4+ T cell counts. Flow cytometry uses lasers to excite fluorescent antibody probes specific for CD4 and other cell surface markers, and to distinguish one type of lymphocyte from another.

Flow cytometry is not cheap. Further, it is technically demanding, complex and costly. Instruments that are commercially available from various manufacturers are significantly expensive, anywhere from $20,000 (USD) TO $95,000 (USD). They can be run only by operators that are sufficiently trained in both the technical and biological aspects of CD4+ T cell. counting. They need special dedicated laboratory space. The counting itself is complex and therefore technically demanding. It requires expensive reagents and regular maintenance if the counts are to be precise and accurate.

All these factors, including the cost of a flow cytometer, technical and operational complexity, the need for reliable electricity, and the high cost of reagents have made the treatment of HIV/AIDS patients a very expensive proposition in all countries, irrespective of whether such countries are third world, resource-poor countries or not. In industrial nations, the factors have pushed insurance costs through the roof and seriously taxed the industrial nations' resources, while in resource-poor countries, these factors have made the use of these instruments impractical and/or difficult to sustain. Thus, the urgent need for affordable and technically simple CD4 diagnostics in both rich and resource-scarce or resource-poor settings is widely recognized, albeit the need is much more pressing in resource-poor settings.

Several efforts have been made to develop alternative, affordable CD4+ T cell counting methods. Single purpose flow cytometers have been designed solely for counting CD4 cells, such as the Becton Dickinson FASCOUNT, the Partec CYFLOW, and desktop instruments from Guava and PointCare Technologies. Low-cost microbead separation of CD4 cells from other blood cells, followed by standard manual cell counting techniques using a light microscope, have also been proposed. Although the former make flow cytometry more affordable in some settings, reagent costs remain high, and the instruments remain expensive, and in most cases, technically complex. While the latter significantly lower reagent costs, as compared to flow cytometry, they are of low throughput, extremely labor intensive, and appear to be less accurate than traditional flow cytometry. Thus, these alternative counting methods do very little to alleviate the depletion of resources and the skyrocketing of insurance costs in industrial countries. Finally, in many developing countries there are few laboratories. Those that do exist are not adequately equipped. Outlying clinics must send samples for testing and wait days for the results, thus losing the opportunity to treat patients by initiating ART, due to the fact that the patients do not return for further treatment once the clinics receive the results.

Thus, the lack of tools for the truly routine, truly inexpensive, speedy, yet effective determination of the need for and implementation of anti-retroviral treatment (ART) needs to be addressed. Treatment with anti-retrovirals, where available, increases the life expectancy of people infected with HIV. Current and future treatment may allow HIV-infected individuals to achieve a life expectancy approaching that of the general public, by proper diagnosis and immediate implementation of ART.

Accordingly, there is a real need for new and better methods and apparatus for assaying CD4+ T cell levels, including methods and apparatus that will avoid the drawbacks of prior art methods and instrumentation while at the same time provide for the assaying of CD4+ T cells at a low cost, without technical and operational complexity, without expensive reagents, without electricity, in a speedy manner while the patient is still present and capable of receiving immediate anti-retroviral therapy, if necessary. Absent such new methods, HIV infection in humans will continue to be pandemic. AIDS will continue to kill both adults and children particularly in resource-poor territories. Such deaths will continue to tax humanity worldwide because they will continue to retard economic growth and increase poverty. Finally, although the need for a simplified and inexpensive assaying of CD4+ T cells count is particularly acute and pressing in resources-poor, third-world countries, such assaying can also be beneficial to industrial countries world wide, by helping to lower their ever increasing and burdensome medical and insurance costs inflicted upon them by AIDS.

In many resource poor settings, in addition to the lack of an inexpensive and easy to perform immune status test typically performed to determine the need for and/or the change of anti-retroviral drug therapy, there are many individuals whose HIV/AIDS status in general have also not been established. Typically, a person who may be suspected of having HIV and/or AIDS is first screened for HIV antibodies using a separate diagnostic test. Once infection status has been determined, these individuals are then monitored, where available, for immune status deterioration requiring the start of an anti-retroviral drug regimen. This can require several visits by the patient, the first to determine whether HIV infection is indeed present and then follow up visit(s) to establish immune status based on the patient's history and symptoms. In rural, resource poor settings, just getting the patients to present themselves for any testing can be problematic, and when results are not available immediately, getting the patient back for follow up can be a daunting task. This can lead to worsening infection, the spread of infection as well as other issues associated with HIV infection and subsequent AIDS, where the body's immune defenses break down and the patient is subject to opportunistic infection and eventually death.

### OBJECTS OF THE INVENTION

It is therefore an object of the present invention to provide a method and apparatus that will put routine immune status testing within the reach of far more HIV patients, particularly in resource-scarce or resource-poor areas.

It is a further object of the present invention to provide a method and apparatus .that will provide and make available an extremely affordable and easy to use rapid diagnostic test for CD4+ T cell levels.

It is an even further object of the present invention to provide a method and apparatus for the determination of CD4+ T cell levels that can be performed without any special instrumentation and which will require no highly skilled personnel, fresh water or electricity.

It is still another object of the present invention to provide a method and apparatus for the determination of CD4+ T cell levels within 20 minutes while the patient is still present, so there is no need for call back, days later, to read test results.

Another object of the present invention is to provide a method and apparatus that will give clinicians the ability to quickly diagnose compromised immune status due to HIV/AIDS while the patient is still in their office and immediately begin ART, if necessary.

It is a further object of the present invention to provide a method and apparatus for providing a cost effective means of monitoring the efficacy of ART and the patient's treatment adherence, even in the most remote of locations.

It is yet another object of the present invention to provide a method and apparatus for the determination of CD4 cell levels at a cost of $5.00 or less versus the current cost of $25-$50 (estimated cost including reagents, equipment, sample transport, etc.), and become accessible to more patients in all settings, helping to optimize ART and the allocation of limited supplies of ART drugs to the patients who need them most.

Another object of the present invention is to provide a method and apparatus that allow the determination of CD4+ T cell levels not by measuring the cells themselves but by measuring CD4+ T cell equivalents, i.e., soluble CD40 ligand, a protein expressed on the surfaces of CD4+ T cells following activation by HIV infection. Such determination will become routine in all countries and provide timely access to anti-retroviral medication even in resource-poor countries.

A further object of the present invention is to provide a method and apparatus that allow the determination of CD4 cell levels without the need for lysis of such cells.

Yet another object of the present invention is to provide a method and apparatus that allow the determination of CD4 cell levels not by measuring the cells themselves, but through a surrogate marker thereof.

It is still another object of the present invention to provide a rapid diagnostic testing device and method which simultaneously detects a patient's HIV/AIDS immune status and detects HIV infection in the patient.

These objects, as well as other objects and advantages, will become apparent from the following disclosure.

### SUMMARY OF THE INVENTION

According to the present invention, there is provided a semi-quantitative, immuno-chromatographic device and method for the simultaneous determination of HIV/AIDS immune status, i.e., via soluble CD40 ligand/CD 154, a CD4+ T cell equivalent protein, and the detection specific antibodies which indicate the presence of HIV infection. It comprises one or more support materials capable of providing lateral flow. As disclosed in co-pending parent application Ser. No. 12/288,180, the one or more support materials of the immunochromatographic strip includes: a) an area for receiving a biological sample containing a target analyte, said analyte being a CD4+ T cell equivalent, namely soluble CD40 ligand/CD 154; (b) an area comprising a movably contained detector ligand, wherein the detector ligand is capable of forming a mobile complex with the soluble CD40 ligand/CD 154; and c) at least one capture area comprising a predetermined amount of an immobile capture reagent, the immobile capture reagent capable of specifically bonding to the mobile complex formed by the soluble CD40 ligand/CD 154 protein and the detector ligand and providing a visible signal. Also, in accordance with the present invention, to detect antibodies which indicate HIV infection, the immunochromatographic device includes at least one additional mobile HIV antibody binding dye complex and an immobile capture area or detection band that is used to indicate the presence of HIV antibodies.

With respect to using the semi-quantitative, immunochromatographic strip 10 for detecting a patient's HIV/AIDS immune status, and as disclosed in co-pending parent application Ser. No. 12/288,180, the method comprises the steps of placing a serum, plasma or whole blood sample at one end of the strip 10; thereafter, allowing the sample to migrate via lateral flow through an area on the strip 10 having an antigen binding monoclonal or polyclonal antibody conjugated to a colloidal gold particle area, which binds with the soluble CD40 ligand/CD 154 in the sample to form a complex; and thereafter, moving the complex, via lateral flow, over a unique combination of monoclonal and/or polyclonal antibodies immobilized on the membrane, which in turn bind the complex to produce a pink/purple band or bands on the strip 10. The remaining complex continues to migrate to a control area on the strip 10, which produces an additional band to show the test has been performed correctly. Each pink or purple band appearing in the test area of the strip 10 corresponds to a number of CD4+ T cell equivalents, which for example can be correlated to one of three categories (see FIGS. 3, 4, 5 and 6): Satisfactory/Normal Immune Status, e.g., about 500 plus CD4+ T cells/μl; Decreasing Immune Status, e.g., less than about 500 plus CD4+ T cells/μl but greater than about 350 CD4+ T cells/μl; or Diminished Immune Status, e.g., less than about 200 CD4+ T cells/μl. Different levels of detection can also be achieved by manipulating the concentrations of the various reagents employed. Furthermore, in accordance with the present invention, a method of detecting and/or quantifying the presence of CD4+ T cells includes the step of detecting and/or quantifying the presence of soluble CD40 ligand/CD 154.

The immunochromatographic device/strip 10 may be simultaneously used for detecting an HIV infection in accordance with steps that are similar to those described above, where the complexed mixture is captured by the at least one additional detection band, which produces a colored line to indicate the presence of HIV antibodies. More specifically, a dual test device is provided. The dual test device determines a patient's HIV/AIDS immune status by measuring soluble CD40 ligand/CD 154, as described previously, but further incorporates a second mobile HIV antibody binding dye complex which binds with any HIV antibodies present in the sample which then migrates along the membrane over an immobilized test line capture area comprised of specific HIV antigens which bind with the anti-HIV antibody dye complex if any HIV antibodies are present forming a visible test line indicating HIV infection. The two dye binding complexes, one each for immune status and HIV infection, can be mixed together or they can be separate depending on the structure of the device, as will be described in greater detail.

In an alternative form of a dual test device, the HIV/AIDS immune status portion of the device may detect and measure the presence of CD4 antigen rather than the CD4+ T Cell equivalent, soluble CD40 ligand (CD154).

By integrating a rapid HIV antibody test into the HIV/AIDS immune status rapid test described above, it would be possible to determine both a person's HIV infection status and simultaneously establish his or her immune status, enabling both to be determined in one visit, while the patient waits. The test would require only a simple addition of a patient sample to the test device. The sample mixes with the reagents incorporated into the device for both HIV antibody and immune status via the previously described HIV/AIDS immune status CD4 equivalent antigen detection device. As the patient sample/reagent mix migrates along the membrane, it passes over two separate test lines containing immobilized detectors, one for HIV antibody detection and one for immune status detection. If either or both of the analytes are present in the sample/reagent mixture, the dye complexed mixture is captured by the immobilized test lines respectively, and a pink/purple band appears, indicating the presence of antibodies to HIV, indicating infection and a semi-quantitative amount of immune-status antigen that correlates to a person's immune status, which can be indicative of requiring ART (anti-retroviral treatment)/HAART (highly active anti-retroviral therapy) or not. The mixture continues to migrate along the membrane to a control line which appears, showing proper performance of the test. This unique combination of tests (HIV infection and immune status) would be both cost effective and easy to perform, requiring no equipment, no highly skilled personnel, no lab facilities, no refrigeration and no clean water, and can be performed on site while the patient is still present and, if results indicate, drug therapy can be initiated or changed, or the patient can be scheduled for further monitoring if he or she is only HIV positive but not yet suffering from the effects of AIDS if his or her immune status is still satisfactory.

### BRIEF DESCRIPTION OF THE DRAWINGS

While the specification concludes with claims which particularly point out and distinctly claim the present invention, it is believed that the present invention will be better understood from the following detailed description taken in conjunction with the accompanying drawings in which like numerals represent identical elements and wherein:

FIG. 1 is a cross-sectional, side view schematic of the inventive semi-quantitative, immunochromatographic strip for the detection of a patient's HIV/AIDS immune status;

FIG. 2 is a top plan view of one of the embodiments of the semi-quantitative, immunochromatographic strip formed in accordance with the present invention for the detection of a patient's HIV/AIDS immune status;

FIGS. 3-6 are top plan views of various embodiments of the semi-quantitative, immunochromatographic strip formed in accordance with the present invention for the detection of a patient's HIV/AIDS immune status and showing CD4+ T cell count equivalents and staging of the immune status.

FIGS. 7a and 7b are respectively an illustrative cross-sectional side view and an illustrative top plan view of a dual test immunochromatographic device for simultaneously detecting, in one test, a patient's HIV/AIDS immune status and an HIV infection in the patient, formed in accordance with a further embodiment of the present invention.

FIGS. 8a and 8b are respectively an illustrative cross-sectional side view and an illustrative top plan view of a dual test immunochromatographic device and strip for simultaneously detecting, in one test, a patient's HIV/AIDS immune status and an HIV infection in the patient, formed in accordance with yet another embodiment of the present invention.

FIG. 9 is an illustrative top plan view of a dual test immunochromatographic strips and device for simultaneously detecting, in one test, a patient's HIV/AIDS immune status and an HIV infection in the patient, formed in accordance with a still further embodiment of the present invention.

LIST OF ELEMENTS AND THEIR RESPECTIVE
IDENTIFYING NUMERALS

| NO. | ELEMENT |
|---|---|
| 10 | Semi-quantitative |
| | Immunochromatographic strip |
| 20 | Support Structure |
| 22 | Top side of the Support Structure 20 |
| 24 | Bottom side of the Support Structure 20 |
| 26 | Proximate end of the Support Structure |
| 28 | Distal end of the Support Structure 20 |
| 30 | Membrane |
| 32 | First end of Membrane |
| 34 | Second end of Membrane |
| 40 | Biological sample receiving area or |
| | sample pad |
| 50 | Control line area |
| 60 | Conjugate pad/detector ligand area |
| 70 | Capture area/Test Line(s) |
| 90 | Sink Pad |
| 100 | Indicia |
| 110 | Housing |
| 120 | Sample Access Port |
| 130 | Viewing Window Cutout |

### DETAILED DESCRIPTION OF THE INVENTION

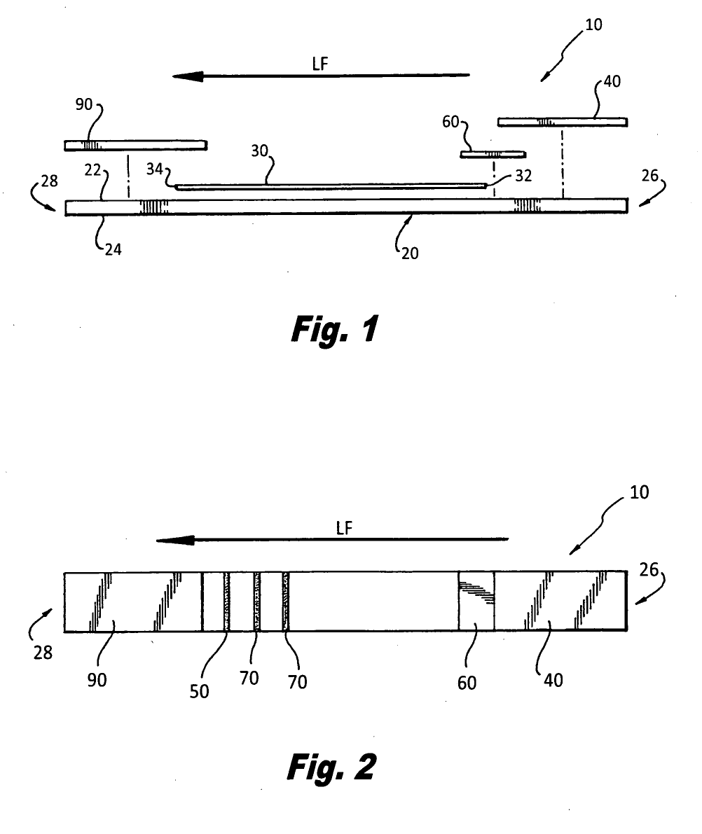

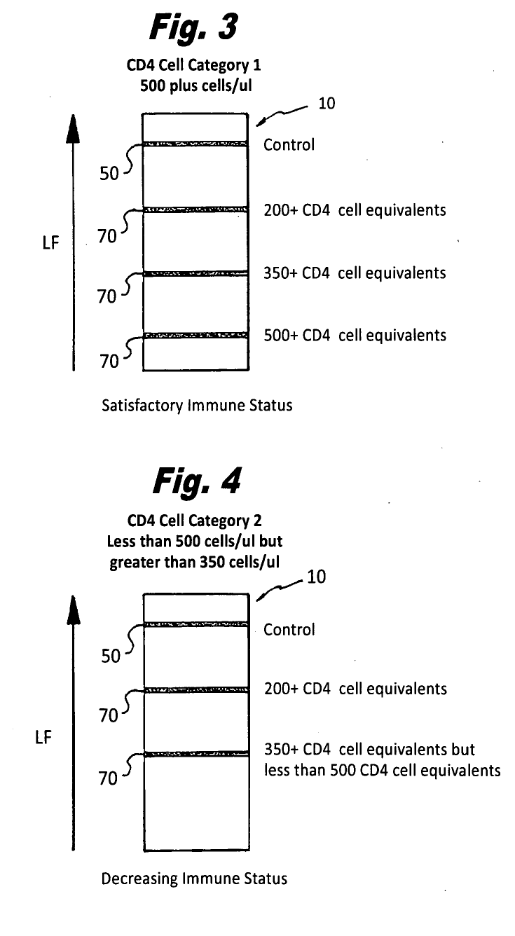

Referring specifically to the drawings, FIGS. 1 and 2 generally depict the inventive semi-quantitative, immunochromatographic test strip and method for use thereof at 10 (hereinafter “the strip 10”). As has been set forth in more detail herein below, it is designed to provide an accurate semi-quantitative, membrane-based screening test for CD4+ T cell levels by assaying a CD4+ T cell equivalent Soluble CD40 ligand/CD 154. It comprises the newest generation of lateral flow immunochromatographic assay devices, which can be used on site with serum, plasma or whole blood samples.

Soluble CD40 ligand/CD 154 is a protein, which is expressed on the surfaces of CD4+ T cells following their activation by HIV infection. The serum levels of this protein have been shown to correlate directly to CD4+ T cell counts. In fact, as disclosed in the article “Levels of Soluble CD40 Ligand (CD 154) in Serum Are Increased in Human Immunodeficiency Virus Type 1-Infected Patients and Correlate with CD4+ T-Cell Counts”, authored by Nikolaos Sipsas, Petros P. Sfikakis, Athanasios Kontos and Theodore Kordossis, published in “Clinical and Diagnostic Laboratory Immunology”, May 2002, p. 558-561, Vol. 9, No. 3, it has been recently determined that the serum levels of this protein appear to be directly proportional to CD4+ T cell counts, making this protein a potentially ideal surrogate marker for determining the immune status of HIV/AIDS patients.

The strip 10 is designed (i) to put routine immune status testing within the reach of far more HIV/AIDS patients, particularly in resource-scarce or resource-poor areas; (ii) to provide and make available an extremely affordable and easy to use rapid diagnostic test for CD4+ T cell levels; (iii) for the determination of CD4+ T cell levels that can be performed without any special instrumentation and which will require no highly skilled personnel, fresh water or electricity; (iv) for the determination of CD4+ T cell levels within 20 minutes while the patient is still present, so there is no need for call back, days or weeks later, to read or interpret test results; (v) to give clinicians the ability to quickly diagnose compromised immune status due to HIV infection while the patient is still present allowing for the immediate start of ART, if necessary; (vi) to provide a cost effective means of monitoring the efficacy of ART and the patient's treatment adherence, even in the most remote of locations; (vii) for the testing of CD4+ T cell levels at a cost of approximately $5.00 or less? versus the current cost of $25-$50 (estimated cost including reagents, equipment, sample transport, etc.); for testing that can be made more accessible to more patients in more settings, helping to optimize ART and the allocation of limited supplies of ART drugs to the patients who need them most; (viii) to allow for the determination of CD4+ T cell levels not by measuring the cells themselves but by measuring CD4+ T cell equivalents, i. e., soluble CD40 ligand/CD 154, and where such determination is envisioned to become routine in all countries and provide timely access to anti-retroviral medication even in resource-poor countries; and (ix) to allow the determination of CD4 cell levels without the need for lysis of such cells.

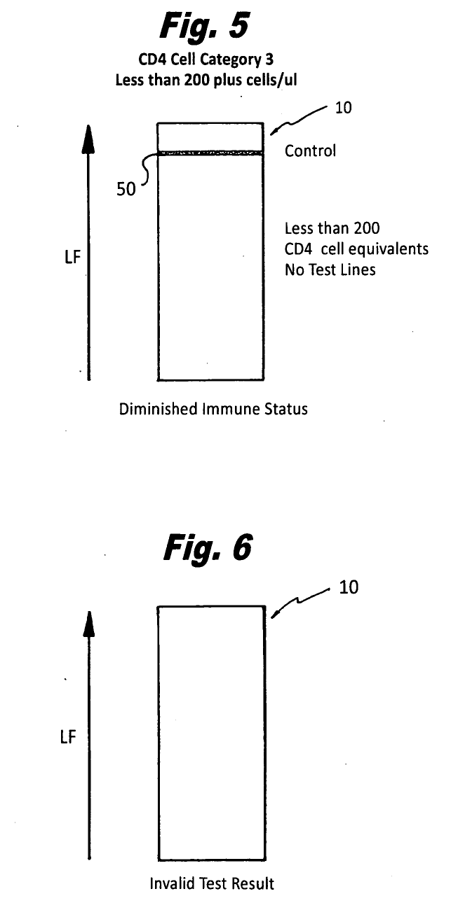

The process of using the semi-quantitative, immunochromatographic strip 10 comprises the steps of placing a serum, plasma or whole blood sample at one end of the strip 10. Thereafter, the sample migrates via lateral flow across the strip 10 through an area containing antigen binding monoclonal or polyclonal antibody conjugated to colloidal gold particles, which binds the soluble CD40 ligand/CD 154 in the sample to form a complex. Thereafter, the complex continues to move via lateral flow over a unique combination of monoclonal and/or polyclonal antibodies immobilized on the strip which in turn bind the complex to generate a pink or purple band or bands on the strip 10. The remaining complex continues to migrate to a control area on the strip 10, which produces an additional band to show that the test has been performed correctly. Each pink or purple band appearing in the test area of the strip 10 corresponds to a number of CD4+ T cell equivalents, which in turn can be correlated for example to one of three categories (see FIGS. 3, 4, 5 and 6): Satisfactory/Normal Immune Status, e.g., about 500 plus CD4+ T cells/μl; Decreasing Immune Status, e.g., less than about 500 plus CD4+ T cells/μl but greater than about 350 CD4+ T cells/μl; or Diminished Immune Status, e.g., less than about 200 CD4+ T cells/μl.

The semi-quantitative immunochromatographic strip 10 comprises a support structure 20 having a top side 22, a bottom side 24, a proximate end 26, and a distal end 28. On said top side 22, at the proximate end 26 of said support structure 20, said semi-quantitative immunochromatographic strip 10 further comprises a biological sample receiving area or sample pad 40; a conjugate pad/detector ligand area 60, immediately adjacent to, downstream from and communicatingly connected to said sample pad 40; a membrane 30 having a first end 32 and a second end 34 opposite to said first end 32, said membrane 30 being immediately adjacent to, downstream from and communicatingly connected to conjugate pad/detector ligand area 60; and a sink pad 90 located at the distal end 28 of the strip 10 immediately adjacent to, downstream from and communicatingly connected to said membrane 30, such that all of the aforementioned components are capable of allowing and not impeding lateral flow, that is, flow in the direction of the arrow LF in FIG. 1.

The semi-quantitative immunochromatographic strip 10 is essentially a composite of its aforementioned components. The steps of forming the composite strip 10, comprise: placing said membrane 30 on said top side 22 of said support structure 20, such that said proximate end 26, and said distal end 28 extend beyond the outer perimeter of the area defined by the placement of said membrane 30 on said support structure 20; placing said conjugate pad/ligand detector 60 on top of the outer edge of first end 32 of said membrane 30 such that part of said conjugate pad/ligand detector area 60 lies partially on top of and is supported by said membrane 30 and part of it extends upstream beyond said membrane and is partially supported by said support structure 20; further placing said sink pad 90 on top of the outer edge of said second end 34 of said membrane 30 such that part of said sink pad 90 lies on top of and is partially supported upstream by said membrane 30 and part of it extends beyond said membrane 30 and is partially supported by said distal end 28 of said support structure 20; and placing said biological sample receiving area or sample pad 40 at said proximate end 26 of said support structure 20 and over said conjugate pad/ligand detector 60 such that part of said sample pad 40 overlaps and is partially supported downstream by said conjugate pad/ligand detector 60 and partially by said proximate end 26 of said support structure 20.

As illustrated in FIG. 2, said support structure 20 can be shaped in the form of a strip. Alternatively, said support structure 20 can be provided in a wide variety of shapes or forms so long as the particular form permits the various functions described herein.

The support structure 20 can be formed from a number of different suitable materials, provided that the materials allow the aforementioned lateral flow functionality. For example, the materials can be comprised of vinyl with adhesive or polyester with adhesive, the adhesive being present to add cohesion to the remaining components of said strip 10 and prevent said strip 10 from falling apart.

The membrane 30 can be formed from a number of different suitable materials so long as such materials allow the aforementioned lateral flow functionality while remaining neutral and unreactive. For example, said membrane 30 can comprise glass fiber, cellulose ester, nylon, cross-linked dextran, etc. According to one embodiment, said membrane 30 comprises nitrocellulose.

As set forth herein above, said membrane 30 has a first end 32 and a second end 34. The edge of said second end 34 extends and fits under said sink pad 90 such that part of said sink pad 90 lies on top of and is partially supported upstream by and communicatingly connected to said membrane 30 and part of it extends beyond said membrane 30 and is partially supported by said distal end 28 of said support structure 20. The edge of said first end 32 extends and fits under said conjugate pad/detector ligand area 60, upstream therefrom and communicatingly connected thereto. Adjacent to said second end 34, but spaced from said sink pad 90, said membrane optionally further comprises a control line area 50. The control line area 50 defines an area which contains a control reagent. The control reagent contained within the control line area 50 is immobile, fixed upon said membrane 30. According to an illustrative non-limiting example, the control reagent is goat anti-mouse IgG.

The control reagent is capable of binding the conjugated detector ligand but is not specific for the soluble CD40 ligand/CD 154. Once it binds the complexed detector ligand, it immobilizes it and continues the lateral flow. Furthermore, a visible colored line appears so as to show that the strip 10 is functioning properly.

In between the control line area 50 and the conjugate pad/detector ligand area 60, said membrane 30 further comprises at least one capture area or test line 70. Alternatively, said membrane 30 can further comprise a plurality of capture areas or test lines 70 (see FIGS. 3, 4 and 5). Each capture area or test line contains a capture reagent therein. The capture reagent is immobily and fixedly contained within said capture area or test line 70. The capture reagent comprises any mouse monoclonal and/or polyclonal anti-sCD40 ligand/CD 154. According to the illustrated embodiments, the capture areas or test lines 70 are distinct and separate from one another in the direction of lateral flow LF. However, it is comprehended by the present invention that one or more capture areas or test lines 70 can be combined and or otherwise merged. Additionally, location, shape, size and configuration of the capture areas/test lines 70 may also deviate from that of the illustrated embodiments.

The biological sample receiving area or sample pad 40 on the proximate end 26 of the device 10 acts as the repository of the patient or biological sample to be tested. It can be formed of cellulose, glass fiber and/or any other material that may also provide for the separation of plasma or serum from whole blood samples. While in the illustrated embodiment only one biological sample receiving area or sample pad 40 is shown located at the proximate end 26 of the strip 10, it should be understood that the present invention can comprise a plurality of sample pads 40 on the proximate end 26 of the strip 10. Moreover, the sample pad 40 can have locations which differ from that of the illustrated embodiment.

The conjugate pad/detector ligand area 60 comprises, optionally, a glass fiber pad, and a detector ligand that is contained within the glass fiber pad in a manner that renders it mobile. In other words, the detector ligand is capable of being carried out of the area 60 by the aforementioned lateral flow. According to an illustrative non-limiting example, a suitably concentrated solution or suspension of detector ligand is applied within area 60 and dried. The detector ligand preferably comprises mouse monoclonal and/or polyclonal anti-sCD40 ligand/CD 154 conjugated to colloidal gold particles. Alternatively, the detector ligand preferably comprises mouse monoclonal and/or polyclonal anti-sCD40 ligand/CD 154 conjugated to colloidal selenium particles; or mouse monoclonal and/or polyclonal anti-sCD40 ligand/CD 154 conjugated to colloidal charcoal particles; or mouse monoclonal and/polyclonal anti-sCD40 ligand/CD 154 conjugated to colloidal latex particles or other suitable colored, fluorescent or magnetic micro particles.

When the detector ligand comes into contact with the soluble CD40 ligand/CD 154 in a biological sample being analyzed, it forms a complex that is also capable of being carried out of the area 60 by lateral flow. When the complex reaches said capture area(s)/test line(s) 70 on said membrane 30, the complex reacts with said capture reagent contained therein and becomes immobily bound to said capture reagent to produce a visible signal, as for example a pink or purple line. When no pink or purple line appears, such as shown in FIG. 6, this could be an indication of an invalid test result.

To the extent that the biological sample contains an amount of soluble CD40 ligand/CD 154 such that the capacity of the capture reagent contained in the capture area/test line 70 is exceeded, any such soluble CD40 ligand/CD 154, and complex formed thereby, continues to travel under the influence of the lateral flow, thereby reaching one or more optionally provided additional capture area/test lines 70.

Controlling the amount of capture reagent contained in the first capture area/test line 70 can be utilized to define a barrier beyond which an amount or concentration of soluble CD40 ligand/CD 154 contained in a biological sample may not pass. To the extent this minimum threshold value is exceeded, excess soluble CD40 ligand/CD 154 is free to travel under the lateral flow to one or more capture areas/test lines 70 which again establish increasing minimum threshold levels of soluble CD40 ligand/CD 154 under analysis. In this regard, the amount of capture reagent provided in each capture area can be the same amount, relative to one another. Alternatively, the amount of capture reagent contained in each capture area can progressively increase or decrease.

The presence of sufficient amount of soluble CD40 ligand/CD 154 and the complex formed from the CD40 ligand/CD 154 and detector ligand in the above-described one or more capture areas/test lines 70 is indicated by the generation of a detectable signal. This detectable signal can be generated in a number of different ways familiar to those skilled in the art. According to one example, the detector ligand can comprise a substance that is immediately and continuously visible to the naked eye. Thus, the mere physical presence of the complex formed between the soluble CD40 ligand/CD 154 and the detector ligand on the one or more capture areas/test lines 70 is sufficient to produce the desired detectable signal. Alternatively, the detector ligand can comprise a first reactant which becomes associated with the complex, formed with the soluble CD40 ligand/CD 154, but which is not visible. A second reactant can then be provided in the one or more capture areas/test lines 70 which upon combination and interaction with the first reactant produces a detectable signal.

In the illustrated embodiment, the area 60 containing the detector ligand and the biological sample receiving area 40 are illustrated as separate and distinct areas on the device 10. However, it is within the scope of the present invention that these two areas could be combined so as to define a single indistinct area of the strip 10.

The strip 10 could also be optionally provided with a housing (not shown). The housing can be formed of any suitable material. For example, the housing may be formed of a plastic material such as Mylar, polystyrene, ABS, etc. The housing at least partially encloses or surrounds the support structure 20. It may be provided with a sample pad window (not shown), a control line area window (not shown) and capture area(s)/test line(s) window(s) (not shown). According to the illustrated embodiments, the area containing the detector ligand is obscured from view of the user by the housing. It is of course comprehended that the area containing the detector ligand may be visible through the housing to the user as well. Moreover, the housing can be formed from a clear or translucent plastic material.

The housing can be optionally provided with indicia which identify various gradations of the concentration of the target CD40 ligand/CD 154 determined to be present in the biological sample by the strip 10. As evident from the indicia illustrated in FIGS. 3, 4 and 5, it is possible to ascertain within a particular range of values the concentration of the CD4+ T cell equivalents, i.e., soluble CD40 ligand/CD 154 present in a biological sample, as described in further detail herein.

The sink pad 90 is made of any material that can act to absorb/stop the lateral flow and permits the lateral flow of the reagents and subsequently absorbs such reagents at the distal end of the test strip after migration 10.

An analysis performed according to the present invention comprises the following steps:
collecting an appropriate biological sample from a patient;
aspirating an appropriate sample volume of the biological sample using a sample pipette. The biological sample can consist of whole blood, serum, or plasma;
depositing the aspirated biological sample to the biological sample receiving area or sample pad 40 of the strip 10;
promoting lateral flow (LF) of the biological sample from the sample pad 40 into and onto the conjugate pad/detector ligand area 60, which contains the detector ligand, e.g., mouse monoclonal and/or polyclonal anti-sCD40 ligand/CD 154 coated on colloidal gold particles, or colloidal selenium particles, or colloidal charcoal particles or colloidal latex particles;
complexing the detector ligand by binding it to the soluble CD40 ligand/CD 154;
moving the complex, via lateral flow, from said conjugate pad/detector ligand area 60 into and onto at least one capture area/test line 70 immobilized on said membrane 30 and containing an immobile capture reagent configured to specifically bind with said complex;
reacting with said complex and immobile capture reagent on said capture area/test line 70 to become immobily bound to the capture reagent and produce a detectably visible signal, such as a pink or purple line; and
once a detectable visible signal is formed on said membrane 30, optionally promoting the lateral flow of whatever detector ligand is left uncomplexed in the biological sample from the capture area/test line 70 to a control line area 50 containing a control reagent which reacts with the uncomplexed detector ligand to produce another visible signal. In this region, the control line area serves as an internal procedural control and the detection of a signal in this area verifies that capillary flow has taken place and that the functional integrity of the device was maintained.

The results of tests performed on HIV/AIDS positive blood samples using the immunochromatographic device of the present invention are provided below in Table I to show the efficacy and operability of the present invention to determine quickly and accurately a patient's immune status by determining the presence of, and quantifying, soluble CD40 ligand/CD 154 in a blood sample. The particular immunochromatographic strip of the present invention used in the performance of these tests was constructed to provide a visible indication (in the form of a band) of soluble CD 40 ligand/CD 154 on the level of about 200 CD4+ T cells/μL or greater.

TABLE I
TEST RESULTS

| | | | Immune- | Immune-STATUS Test Results | |
|---|---|---|---|---|---|
| | | | STATUS Rapid | Ranked as Positive (>200 CD4 | |
| | | | Test Type | EQ's) or Negative (<200 CD4 | |
| | | | Immune- | EQ's) | |
| | | | STATUS Quick | NOTE: | |
| Sample | CD4 | Viral | Screen | Negative = No Test Line | Date |
| Number | Count | Load | CD4 EQ 200 | Positive = Any visible Test Line | Run |
| 0005-041- | 1000 | | QS200 | Positive | Jul. 01, 2007 |
| 01472 | | | | | |
| 0006-041- | 225 | | QS200 | Negative | Jul. 01, 2007 |
| 01825 | | | | | |
| 0008-041- | 174 | | QS200 | Negative | Jul. 01, 2007 |
| 00640 | | | | | |
| 0008-214- | 528 | | QS200 | Positive | Jul. 01, 2007 |
| 02462 | | | | | |
| 0009-041- | 354 | | QS200 | Positive | Jul. 01, 2007 |
| 01357 | | | | | |
| 0006-041- | 994 | 63 | QS200 | Positive | Sep. 05, 2007 |
| 00754 | | | | | |
| 0008-041- | 599 | 188 | QS200 | Positive | Sep. 05, 2007 |
| 01001 | | | | | |
| 0008-214- | 200 | 5730 | QS200 | Positive | Sep. 05, 2007 |
| 02455 | | | | | |
| 0009-041- | 554 | <25 | QS200 | Positive | Sep. 05, 2007 |
| 01333 | | | | | |
| 0009-041- | 376 | <50 | QS200 | Positive | Sep. 05, 2007 |
| 01345 | | | | | |
| 0010-258- | 400 | 1640 | QS200 | Positive | Sep. 05, 2007 |
| 02458 | | | | | |
| 0006-041- | 402 | <50 | QS200 | Positive | Sep. 24, 2007 |
| 01826 | | | | | |
| 0009-096- | 451 | 800 | QS200 | Positive | Sep. 24, 2007 |
| 01781 | | | | | |
| 0009-214- | 501 | <50 | QS200 | Positive | Sep. 24, 2007 |
| 01609 | | | | | |
| 0010-214- | 456 | 15679 | QS200 | Positive | Sep. 24, 2007 |
| 00041 | | | | | |
| 0005-041- | 278 | 13211 | QS200 | Positive | Nov. 06, 2007 |
| 01454 | | | | | |
| 0005-041- | 301 | 6629 | QS200 | Positive | Nov. 06, 2007 |
| 01695 | | | | | |
| 0006-041- | 254 | 26801 | QS200 | Positive (very weak) | Nov. 06, 2007 |
| 00772 | | | | | |
| 0006-041- | 256 | <50 | QS200 | Positive (very weak) | Nov. 06, 2007 |
| 01524 | | | | | |
| 0007-041- | 205 | 70 | QS200 | Negative | Nov. 06, 2007 |
| 01775 | | | | | |
| 0008-041- | 226 | 4972 | QS200 | Negative | Nov. 06, 2007 |
| 00993 | | | | | |
| 0009-214- | 301 | 39652 | QS200 | Positive | Nov. 06, 2007 |
| 01940 | | | | | |
| 145719 | 1165 | N/A | QS200 | Positive | Oct. 08, 2007 |
| 145721 | 920 | N/A | QS200 | Positive | Oct. 08, 2007 |
| 145723 | 1012 | N/A | QS200 | Positive | Oct. 08, 2007 |
| 145725 | 578 | N/A | QS200 | Positive | Oct. 08, 2007 |
| 145726 | 641 | N/A | QS200 | Positive | Oct. 17, 2007 |
| 146106 | 923 | N/A | QS200 | Positive | Oct. 17, 2007 |
| 146108 | 880 | N/A | QS200 | Positive | Oct. 17, 2007 |
| IN 01 | 457 | N/A | QS200 | Positive | Dec. 06, 2007 |
| IN 02 | 142 | N/A | QS200 | Positive (very weak)* | Dec. 06, 2007 |
| IN 03 | 304 | N/A | QS200 | Positive | Dec. 06, 2007 |
| IN04 | 604 | N/A | QS200 | Positive | Dec. 20, 2007 |
| IN05 | 287 | N/A | QS200 | Negative | Dec. 20, 2007 |
| IN06 | 99 | N/A | QS200 | Positive (very weak)* | Dec. 20, 2007 |
| IN07 | 214 | N/A | QS200 | Positive (very faint) | Dec. 20, 2007 |
| IN08 | 537 | N/A | QS200 | Positive | Dec. 20, 2007 |
| IN09 | 634 | N/A | QS200 | Positive | Dec. 20, 2007 |
| IN10 | 225 | N/A | QS200 | Positive (weak line) | Dec. 20, 2007 |
| IN11 | 32 | N/A | QS200 | Positive* | Dec. 20, 2007 |
| IN12 | 171 | N/A | QS200 | Positive (very faint line)* | Dec. 20, 2007 |
| IN13 | 193 | N/A | QS200 | Positive (very faint line)* | Dec. 20, 2007 |
| IN14 | 395 | N/A | QS200 | Positive | Dec. 20, 2007 |
| IN15 | 384 | N/A | QS200 | Positive | Dec. 20, 2007 |
| IN16 | 103 | N/A | QS200 | Negative | Dec. 20, 2007 |
| IN17 | 639 | N/A | QS200 | Positive | Dec. 20, 2007 |
| SA1 | 133 | N/A | QS200 | Positive* | Mar. 10, 2008 |
| SA2 | 176 | N/A | QS200 | Positive | Mar. 10, 2008 |
| SA3 | 211 | N/A | QS200 | Positive | Mar. 10, 2008 |
| SA4 | 222 | N/A | QS200 | Positive | Mar. 10, 2008 |

TABLE I - COMMENTS
*Results marked with an asterisk (*) are discordant.
All results at 200 +/− 25 CD4 cells were considered positive based on the tests low end cutoff
N = 50 (As of March 2008)
Correlation to Flow Cytometry = 89.1%

Results for the rapid test performed with the immunochromatographic device of the present invention are reported in Table I as either “positive” when a colored band of any intensity is seen in the test area of the device or “negative” when no colored band is visible in the test area. As the amount of CD4 equivalent proteins approaches the dynamic cutoff for the test which is approximately 200+/−25 CD4 cells, the colored band in the test area of the device will become lighter and lighter until it disappears at approximately 200+/−25 CD4 cells.

The immunochromatographic device of the present invention provides a one step screening test for the semi-quantitative determination of immune status in HIV/AIDS patients via detection of specific proteins that directly correlate to CD4 counts (CD4 equivalents). To reiterate, AIDS is characterized by changes in the amount of T-cell lymphocytes. The virus, in infected individuals, causes a depletion of the T-helper cells, which are a sub population of T-cells. This leaves patients susceptible to opportunistic infections and potential malignancies. The presence of the virus itself causes the immune system to deteriorate as AIDS progresses. In normal and immune suppressed individuals, there are specific proteins that are detectable that correlate to CD4 counts and which can be detected by the device and method of the present invention, as evidenced by the test results shown in Table I. Tests such as CD4 counts are among the most widely used method for determining the immune status of HIV/AIDS infected patients and to establish the efficacy and/or timing of the start of ART (anti-retroviral therapy).

The present invention provides a rapid membrane based screening test to detect the presence of specific proteins that correlate to CD4 counts. This test is the newest generation lateral flow immunochromatographic type assay. These are among the simplest and easiest to use POC (point of care) assays requiring no instrumentation or highly skilled individuals to perform. Test using the device of the present invention can be performed using fresh plasma or serum samples. The test employs the use of an antigen binding monoclonal antibody conjugated to a colloidal gold particle and a unique combination of monoclonal antibodies immobilized on the membrane.

As described previously, once the sample is added to the test device, the mixture passes through the antigen binding/gold complex, which then binds the specific target protein in the sample. As this complex passes over the immobilized antibodies on the membrane, if any specific CD4 equivalent proteins are present, the antibodies capture them in turn. This produces a pink/purple band or band(s) in a particular area of the test device. The remaining complex continues to migrate to a control area on the test device and produces a pink/purple band in this control area. This control band indicates that the test has been performed properly.

As a result of the components of the inventive device described herein and its various embodiments disclosed herein above and the way they cooperatingly function, it is clear that they achieve all of the objectives set forth herein above including: (i) putting routine immune status testing within the reach of far more HIV patients, particularly in resource-scarce or resource-poor areas; (ii) providing and making available an extremely affordable and easy to use rapid diagnostic test for CD4+ T cell levels; (iii) the determination of CD4+ T cell levels that can be performed without any special instrumentation and which will require no highly skilled personnel, fresh water or electricity; (iv) the determination of CD4+ T cell levels within 20 minutes while the patient is still present, so there is no need for call back, days later, to read test results; (v) giving clinicians the ability to quickly diagnose compromised immune status due to HIV/AIDS while the patient is still in their office and immediately begin ART, if necessary; (vi) providing a cost effective means of monitoring the efficacy of ART and the patient's treatment adherence, even in the most remote of locations; (vii) the testing of CD4 cell levels at a projected cost of approximately $5.00 versus the current cost of $25-$50 (estimated cost including reagents, equipment, sample transport, etc.); (viii) allowing for the determination of CD4+ T cell levels not by measuring the cells themselves but by measuring CD4+ T cell equivalents, i. e., soluble CD40 ligand; and (ix) allowing the determination of CD4 cell levels without the lysis of such cells.

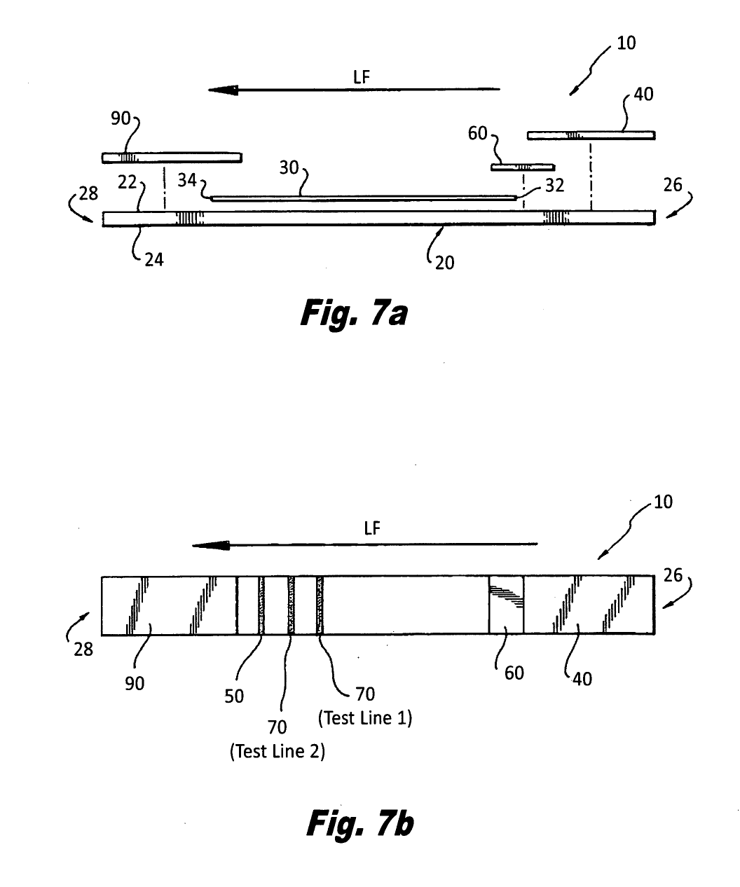

FIGS. 7-9 show various embodiments of a dual test immunochromatographic strip 10 for simultaneously detecting, in one test, a patient's HIV/AIDS immune status and the presence of an HIV infection in the patient, formed in accordance with the present invention. Each of the dual test immunochromatographic strips 10 illustrated by FIGS. 7-9 include the structure of the single test, HIV/AIDS immune status immunochromatographic device described previously herein and illustrated by FIGS. 1-6 of the drawings. However, with the dual test strip 10, there is included at least one additional HIV antibody binding mobile complex and a separate capture area comprised of a predetermined amount of an immobile capture reagent (also referred to herein as a detection band or test line).

One form of the dual test strip 10 is shown in FIGS. 7a and 7b. A supporting substrate 20 provides support for the various areas and membranes described below. An area 40 is provided on the substrate 20 for receiving a biological sample containing the target analyte or analytes. Another area 60 is provided on the substrate 20 overlapping the sample receiving area 40 and the membrane 30 that comprises a movably contained detector ligand which, as described previously, is capable of forming a mobile complex with soluble CD40 ligand/CD 154, which is at least one of the target analytes in the biological sample. The area 60 further includes a mobile HIV antibody binding dye complex which binds with any HIV antibodies present in the sample which migrates along the membrane 30. At least one capture area 70 (shown as Test Line 2 in FIG. 7b) is used for testing the HIV/AIDS immune status of the patient. This. capture area 70 comprises a predetermined amount of an immobile capture reagent which is capable of specifically bonding to the mobile complex formed by the soluble CD40 ligand/CD 154 protein and the detector ligand and provides a visible indication preferably in the form of a pink/purple band. At least one additional capture area 70 (shown in FIG. 7b as Test Line 1), which is separate from the immune status capture area 70 (shown in FIG. 7b as Test Line 2) includes an amount of immobile capture reagents which causes the sample/reagent mixture migrating along the membrane 30 to bond thereto if an HIV antibody is present in the patient's blood or serum sample. This additional detection band 70 (i.e., Test Line 1) will turn a pink/purple color, which indicates the presence of antibodies to HIV.

More specifically, the patient sample placed on the dual test strip 10 mixes with the reagents incorporated into the device for both HIV antibody and CD40 equivalent antigen detection. As the patient sample/reagent mix migrates along the membrane 30, it passes over the at least two separate test lines 70 containing immobilized detectors, at least one for HIV antibody detection and at least one for immune status detection. If either or both of the analytes are present in the sample/reagent mixture, the dye complex mixture is captured by the immobilized test lines 70 respectively, and a pink/purple band appears, indicating the presence of antibodies to HIV, indicating infection and a semi-quantitative amount of immune-status antigen that correlates to a person's immune status, which can be indicative of requiring ART/HAART treatment or not. The mixture continues to migrate along the membrane to a control line 50 which appears, showing proper performance of the test. It should be noted that in the embodiment of the dual test device described above and shown in FIG. 7, the two dye binding complexes, one each for immune status and HIV infection, are mixed together.

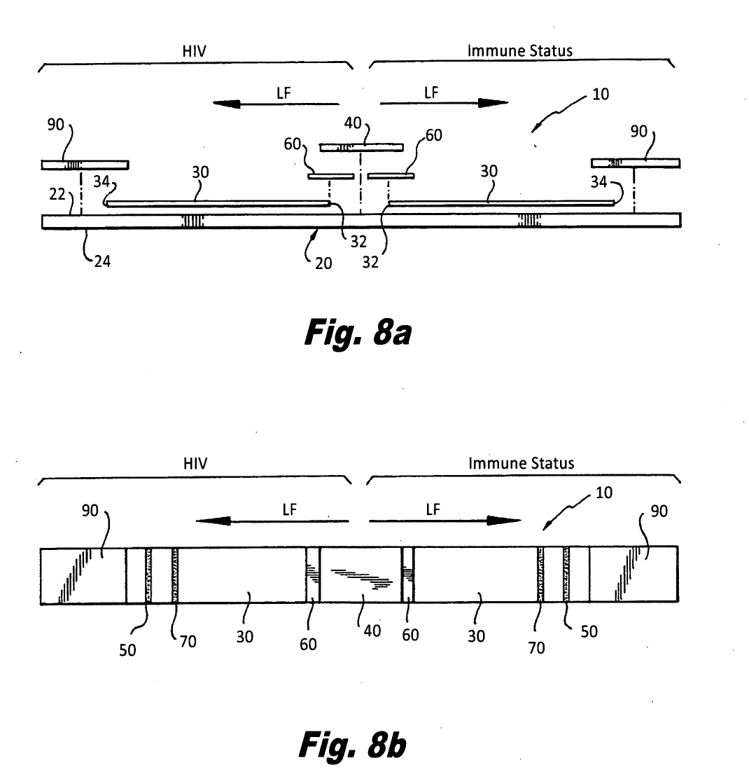

Another alternative form of a dual test device 10 formed in accordance with the present invention is shown in FIGS. 8a and 8b of the drawings. This is a multi-membrane configuration, whereby the two tests, HIV and immune status, are on separate membranes 30 but incorporated into a single device to provide bi-directional flow. The sample receiving area 40 is situated centrally on the elongated supporting substrate 20, and the two separate membranes 30 extend from the sample receiving area 40 in opposite directions on the substrate 20 and are in fluid communication with the sample receiving area 40 either directly or through an interposed conjugate pad/detector ligand area 60. At least one test line or capture area 70 for HIV antibody detection is situated on the first membrane 30, and a control line or area 50 is situated on the first membrane 30 at a position on the first membrane preferably farther away from the sample receiving area 50 than the HIV antibody test line 70.

Similarly, the second membrane 30, also in fluid communication with the sample receiving area 40 either directly or through an interposed conjugate pad/detector ligand area 60, which is used for HIV/AIDS immune status determination, includes at least one test line or capture area 70 having a predetermined amount of an immobile capture reagent capable of specifically bonding to the mobile complex formed by the soluble CD40 ligand/CD154 protein and the detector ligand on the second membrane 30 to provide a visible signal indicative of the presence and/or quantity of HIV/AIDS immune status CD4+ T cell equivalents. A control line or area 50 is also provided on the second membrane 30, which is disposed on the second membrane 30 preferably farther away from the sample receiving area 40 than the immune status test line 70. Both control lines or areas 50 will change colors to indicate that the tests have been performed properly, as described previously. With the dual test device 10 shown in FIG. 8, fluid flow is bi-directional, in opposite directions, from a single sample receiving area 40, with the HIV antibody test section on one side of the sample receiving area 40, and the HIV/AIDS immune status test area on the diametrically opposite side of the sample receiving area 40. Each of the first membrane 30 and the second membrane 30 is in fluid communication with the sample receiving area 40.

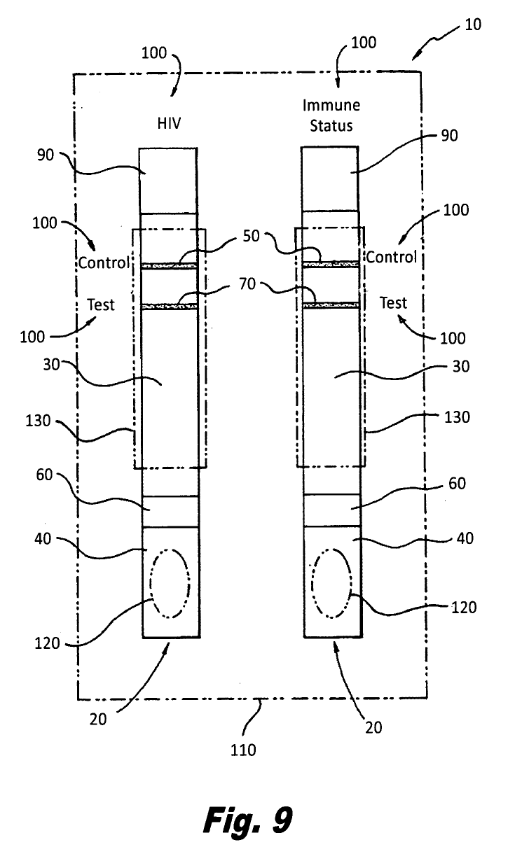

Another version of the dual test device 10 formed in accordance with the present invention is shown in FIG. 9 of the drawings. Here, a multi-membrane configuration is used, whereby the two tests, HIV and immune status, are performed on separate parallelly disposed membranes 30 on the overall supporting substrate 20 of the device but, as can be seen, are incorporated into a single device within a housing 110. Two separate sample receiving areas 40, each being respectively in fluid communication with a corresponding membrane 30 either directly or through an interposed conjugate pad/detector ligand area 60, are provided on the substrate 20 of the device. Each sample receiving area 40 is exposed through a respective sample access port 120 formed through the thickness of the housing 110. Similarly, at least a portion of each membrane 30 is exposed through a viewing window formed as a cutout 130, with or without a transparent covering (not shown) on each cutout 130, through the thickness of the housing 110. As described previously with respect to the bi-directional flow dual test device 10 shown in FIGS. 8a and 8b, the parallel flow dual test device 10 shown in FIG. 9 has at least one test line or capture area 70 and a control line or area 50 provided on each of the membranes 30. The at least one test line 70 on one of the membranes 30 is provided for detecting the presence of an HIV antibody in the patient sample, and the at least one test line 70 on the other membrane 30 is provided for detecting and/or quantifying the patient's HIV/AIDS immune status.

As can be seen from FIG. 9 of the drawings, the dual test device 10 may include labels or other indicia 100 situated in proximity to the HIV test portion and the immune status test portion so that the physician or technician administering the test will know which portion of the dual test device 10 relates to HIV detection and which portion of the dual test device 10 relates to immune status detection.

A rapid, dual test device formed in accordance with the present invention may, instead of detecting and measuring the presence of a CD4+ T cell equivalent, that is, soluble CD40 ligand (CD154), directly detect and measure the presence of CD4 cell antigen to determine a patient's HIV/AIDS immune status. The HIV antibody detection portion of the dual test device would remain the same as described previously.

The unique combination of tests (HIV infection and immune status) situated on a single device would be both cost effective and easy to perform, requiring no equipment, no highly skilled personnel, no lab facilities and no clean water, and can be performed on site while the patient is still present and, if results indicate, drug therapy can be initiated or changed, or the patient can be scheduled for further monitoring if he or she is only HIV positive but not yet suffering from the effects of AIDS if his or her immune status is still satisfactory.

While particular embodiments of the invention have been illustrated and described in detail herein, they are provided by way of illustration only and should not be construed to limit the invention. Since certain changes may be made without departing from the scope of the present invention, it is intended that all matter contained in the above description or shown in the accompanying drawings be interpreted as illustrative and not in a literal sense. Practitioners of the art will realize that the sequence of steps and the embodiments depicted in the figures can be altered without departing from the scope of the present invention and that the illustrations contained herein are singular examples of a multitude of possible depictions of the present invention.

What is claimed is:

1. A dual test device for the simultaneous detection of an HIV/AIDS immune status CD4+ T cell equivalent and an HIV antibody, which comprises:
one or more support materials capable of providing lateral flow;
at least a first sample receiving area situated on at least one of the one or more support materials for receiving a biological sample containing at least a first target analyte and a second target analyte, the first target analyte being a CD4+ T cell equivalent, the second target analyte being an HIV antibody;
at least one second area situated on at least one of the one or more support materials having a movably contained detector ligand conjugate, wherein the detector ligand is capable of 10 forming a mobile complex with the CD4+ T cell equivalent;
at least a first capture area situated on at least one of the one or more support materials having a predetermined amount of a first immobile capture reagent, the first immobile capture reagent capable of specifically binding to the mobile complex formed by the CD4+ T cell equivalent protein and the detector ligand and providing a visible signal; and
at least a second capture area situated on at least one of the one or more support materials having a predetermined amount of a second immobile capture reagent capable of specifically binding with an HIV antibody present in the biological sample.

2. A dual test device as defined by claim 1, wherein the CD4+T cell equivalent is soluble CD40 ligand/CD 154.

3. A dual test device as defined by claim 2, wherein the detector ligand of the at least one second area includes at least one of mouse monoclonal and/or polyclonal anti-sCD40 ligand/CD 154 conjugated to colloidal gold particles; mouse monoclonal and/or polyclonal anti-sCD40 ligand/CD 154 conjugated to colloidal selenium particles; mouse monoclonal and/or polyclonal anti-sCD40 ligand/CD 154 conjugated to colloidal charcoal particles; and mouse monoclonal and/or polyclonal anti-sCD40 ligand/CD 154 conjugated to colloidal latex particles.

4. A dual test device as defined by claim 2, wherein the first immobile capture reagent situated on the at least one first capture area includes a mouse monoclonal and/or polyclonal anti-sCD40 ligand/CD 154.

5. A dual test device as defined by claim 1, which further comprises:
at least a first control area situated on at least one of the one or more support materials, the at least first control area including an immobile control reagent providing a visible indication and which is capable of binding the conjugated detector ligand but does not recognize or bind to the CD4+ T cell equivalent, whereupon the control reagent binds the conjugated detector ligand and changes color so as to show that the device is working properly.

6. A dual test device as defined by claim 5, wherein the control reagent includes goat anti-mouse IgG.

7. A dual test device as defined by claim 1, which further comprises:
at least a first control area situated on at least one of the one or more support materials, the at least first control area including an immobile control reagent providing a visible indication so as to show that the device is working properly with respect to at least one of an HIV/AIDS immune status test and an HIV antibody detection test.

8. A dual test device as defined by claim 1, which further comprises:
at least a first control area and a second control area, the at least first control area and the second control area being situated on at least one of the one or more support materials, the at least first control area including a first immobile control reagent providing a visible indication so as to show that the device is working properly with respect to an HIV/AIDS immune status test, the at least second control area including a second immobile control reagent providing a visible indication so as to show that the device is working properly with respect to an HIV antibody detection test.

9. A dual test device as defined by claim 2, wherein the detector ligand includes a first reactant which becomes associated with the complex formed with the soluble CD40 ligand/CD 154, and a second reactant situated on the at least first capture area which upon binding and interaction with the first reactant produces a detectable signal.

10. A dual test device for the simultaneous detection of an HIV/AIDS immune status CD4+ T cell equivalent and an HIV antibody, which comprises:
a support material capable of providing lateral fluid flow;
a sample receiving area situated on the support material for receiving a biological sample containing at least a first target analyte and a second target analyte, the first target analyte being a CD4+ T cell equivalent, the second target analyte being an HIV antibody;
at least one second area situated on the support material and in fluid communication with the sample receiving area, the at least one second area having a movably contained detector ligand conjugate, wherein the detector ligand is capable of forming a mobile complex with the CD4+ T cell equivalent;
at least a first capture area situated on the support material and being in fluid communication with the sample receiving area, the first capture area having a predetermined amount of a first immobile capture reagent, the first immobile capture reagent being capable of specifically binding to the mobile complex formed by the CD4+ T cell equivalent protein and detector ligand and providing a visible signal;
at least a second capture area situated on the support material and being in fluid communication with the sample receiving area, the second capture area having a predetermined amount of a second immobile capture reagent capable of specifically binding to an HIV antibody present in the biological sample and providing a visible signal; and
at least one control area situated on the support material, the at least one control area including an immobile control reagent providing a visible indication so as to show that the device is working properly with respect to at least one of an HIV/AIDS immune status test and an HIV antibody detection test.

11. A dual test device for the simultaneous detection of an HIV/AIDS immune status CD4+ T cell equivalent and an HIV antibody, which comprises:
a support material capable of providing lateral fluid flow;
a first sample receiving area and a second sample receiving area, each of the first sample receiving area and the second sample receiving area being situated on the support material and being provided for receiving a biological sample containing at least a first target analyte and a second target analyte, the first target analyte being a CD4+ T cell equivalent, the second target analyte being an HIV antibody;
at least one second area situated on the support material and in fluid communication with the first sample receiving area, the at least one second area having a moveably contained detector ligand conjugate, wherein the detector ligand is capable of forming a mobile complex with the CD4+ T cell equivalent;
at least a first capture area situated on the support material and being in fluid communication with the first sample receiving area, the first capture area having a predetermined amount of a first immobile capture reagent, the first immobile capture reagent capable of specifically binding to the mobile complex formed by the CD4+ T cell equivalent protein and detector ligand and providing a visible signal;
at least a second capture area situated on the support material and being in fluid communication with the second sample receiving area, the second capture area having a predetermined amount of a second immobile capture reagent capable of specifically binding to an HIV antibody present in the biological sample;
a first control area situated on the support material, the first control area including a first immobile control reagent providing a visible indication so as to show that the device is working properly with respect to an HIV/AIDS immune status test; and
a second control area situated on the support material, the second control area including a second immobile control reagent providing a visible indication so as to show that the device is working properly with respect to an HIV antibody detection test.

12. A dual test device for the simultaneous detection of an HIV/AIDS immune status CD4+ T cell equivalent and HIV antibody, which comprises:
a support material capable of providing lateral fluid flow;
a sample receiving area, the sample receiving area being situated on the support material and being provided for receiving a biological sample containing at least a first target analyte and a second target analyte, the first target analyte being a CD4+ T cell equivalent, the second target analyte being an HIV antibody;
at least one second area situated on the support material and in fluid communication with the sample receiving area, the at least one second area having a moveably contained detector ligand conjugate, wherein the detector ligand is capable of forming a mobile complex with the CD4+ T cell equivalent;
at least a first capture area situated on the support material and being in fluid communication with the sample receiving area, the first capture area having a predetermined amount of a first immobile capture reagent, the first immobile capture reagent capable of specifically binding to the mobile complex formed by the CD4+ T cell equivalent protein and detector ligand and providing a visible signal;
at least a second capture area situated on the support material and being in fluid communication with the sample receiving area, the second capture area having a predetermined amount of a second immobile capture reagent capable of specifically binding to an HIV antibody present in the biological sample;
a first control area situated on the support material, the first control area including a first immobile control reagent providing a visible indication so as to show that the device is working properly with respect to an HIV/AIDS immune status test; and
a second control area situated on the support material, the second control area including a second immobile control reagent providing a visible indication so as to show that the device is working properly with respect to an HIV antibody detection test.

13. A method of using a semi-quantitative, immunochromatographic device for the simultaneous detection of an HIV/AIDS immune status CD4+ T cell equivalent and an HIV antibody, which comprises the steps of:
placing a serum, plasma or whole blood sample at one end of the device on at least one sample receiving area on the device that contains at least a first target analyte and a second target analyte, the first target analyte being a CD4+ T cell equivalent, the second target analyte being an HIV antibody;
moving the sample, via lateral flow, to at least a second area on the device that contains a conjugated detector ligand capable of forming a mobile complex with the CD4+ T cell equivalent;
moving the complex, via lateral flow, to at least a first capture area on the device having a predetermined amount of a first immobile capture reagent capable of binding to the mobile complex formed by the CD4+ T cell equivalent and the conjugated detector ligand and providing a visible indication; and
moving one of the sample and the complex, via lateral flow, to at least a second capture area on the device having a predetermined amount of a second immobile capture reagent capable of binding to an HIV antibody present in the sample and providing a visible indication.

14. A method as defined by claim 13, wherein the CD4+ T cell equivalent is soluble CD40 ligand/CD 154.

15. A method as defined by claim 13, which further comprises the steps of:
moving the complex, via lateral flow, over the at least first capture area having the predetermined amount of the first immobile capture reagent, allowing a remaining portion of the complex to continue to flow to a first control area on the device having a first control reagent situated thereon which produces a visible indication that the method has been performed correctly.

16. A method as defined by claim 15, which further comprises the step of:
moving one of the sample and the complex, via lateral flow, over the at least second capture area having the predetermined amount of the second immobile capture reagent, allowing a remaining portion of one of the sample and the complex to continue to flow to a second control area on the device having a second control reagent situated thereon which produces a visible indication that the method has been performed correctly.

17. A method as defined by claim 13, wherein the detector ligand is at least one mouse monoclonal and/or polyclonal anti-sCD40 ligand/CD 154 conjugated to colloidal gold particles; mouse monoclonal and/or polyclonal anti-sCD40 ligand/CD 154 conjugated to colloidal selenium particles; mouse monoclonal and/or polygonal anti-sCD40 ligand/CD 154 conjugated to colloidal charcoal particles; and mouse monoclonal and/or polyclonal anti-sCD40 ligand/CD 154 conjugated to colloidal latex particles.

18. A method as defined by claim 13, wherein the first capture reagent includes a mouse monoclonal and/or polyclonal anti-sCD40 ligand/CD 154.

19. A method as defined by claim 13, which further comprises the step of detecting or quantifying the presence of soluble CD40 ligand/CD 154.

20. A dual test device for the simultaneous detection of an HIV/AIDS immune status CD4+ T cell equivalent and an HIV antibody, which comprises:
one or more support materials capable of providing lateral flow;
at least a first sample receiving area situated on at least one of the one or more support materials for receiving a biological sample containing at least a first target analyte and a second target analyte, the first target analyte being a CD4 antigen, the second target analyte being an HIV antibody;
at least one second area situated on at least one of the one or more support materials having a movably contained CD4 antigen detector, wherein the detector is capable of forming a mobile complex with the CD4 antigen;
at least a first capture area situated on at least one of the one or more support materials having a predetermined amount of a first immobile capture reagent, the first immobile capture reagent capable of specifically binding to the mobile complex formed by the CD4 antigen and the CD4 antigen detector and providing a visible signal; and
at least a second capture area situated on at least one of the one or more support materials having a predetermined amount of a second immobile capture reagent capable of specifically binding with an HIV antibody present in the biological sample.
